(19) United States
(12) Patent Application Publication
(10) Pub. No.: US 2026/0263769 A1
(43) Pub. Date: Sep. 10, 2026

(54) INTEGRATED TOOL TO INSERT PELLET-RELEASING BIOACTIVE COMPOUND(S) IN A REGION TO BE TREATED OF A SUBJECT IN NEED

(71) Applicant: PALTECH, Paris (FR)

(72) Inventors: Charles-Henri MALBERT, Bruz (FR); Maurice Reginald ALLOUCHE, Paris (FR)

(73) Assignee: PALTECH, Paris (FR)

(21) Appl. No.: 19/473,090

(22) PCT Filed: Apr. 7, 2023
(86) PCT No.: PCT/IB2023/000187

§ 371 Date: Oct. 6, 2025

(51) Int. Cl. A61M 37/00; A61B 17/3203; A61B 18/00; A61B 18/14

(52) U.S. Cl. CPC A61M 37/0069; A61B 18/1482; A61B 18/1492; A61B 17/3203; A61B 2018/0091; A61B 2018/1475

(57) ABSTRACT

The invention concerns a laparoscopic tool (10) comprising:•—a shaft (102) comprising a proximal end and a distal end;•—a tip (100);•—a moving part (101) disposed between the distal end of the shaft and the tip, the moving part being attached to both the distal end and the tip, the moving part being adapted to allow movement of the tip relative to the shaft; wherein the tip comprises a cylindrical part and a tapered part extending from the cylindrical part to an outlet; the cylindrical part being attached to the moving part; the cylindrical part comprising a central channel surrounded by at least two peripheral channels; the central channel and the peripheral channels extending across the cylindrical part of the tip wherein the central channel comprises a pellet (20) together with a foldable retainer in a folded position; the first peripheral channel comprising an opening at the tapered part, the first peripheral channel comprising a first dissection tool that is capable to be protruded from the tip; a blinded peripheral channel comprising a location sensor (30) configured to be detected by an external localizer (32) for locating continuously the tip.

### TECHNICAL FIELD

The present disclosure relates to a surgical tool in particular a laparoscopic tool designed to insert a pellet of a bioactive molecule in a region to be treated. The present disclosure also relates to a surgical method using such a tool.

### BACKGROUND

Several surgical steps are mandatory to achieve a positive outcome of a treatment using locally implanted bioactive molecule (in a pellet formulation for instance), all of which require sequential insertion of dedicated laparoscopic tools. Furthermore, in order to administer the molecule precisely, the region to be treated being often narrow, a complex image guided procedure is needed with continuous 3D localization of the tip of the instruments.

Therefore, such a procedure involves several tools to be inserted or removed from the patient. This multiplicity of insertions increases injury risk for the patient (especially at the vicinity of critical organs e.g., liver or vascular beds). Finally, it is necessary to ensure that the pellet remains in the region to be treated once it has been inserted at the correct anatomical location. This last step requires further interventions and additional tools.

Therefore, there is a need to have a single tool suitable for laparoscopy and capable of depositing a bioactive molecule in a targeted region to be treated without extraction and reinsertion of multiple tools in the abdominal cavity of the patient. Furthermore, because this tool is unique, its moveability must be greater than a single laparoscopic forceps making mandatory a capacity for remote steering of the terminal end.

### SUMMARY

The present application aims at providing a single surgical tool suitable for laparoscopy surgery and capable of achieving several tasks without extraction and reinsertion of the tool in the patient.

To this end the present disclosure concerns a laparoscopic tool comprising:
a shaft comprising a proximal end and a distal end;
a tip;
a moving part disposed between the distal end of the shaft and the tip, the moving part being attached to both the distal end and the tip, the moving part being adapted to allow movement of the tip relative to the shaft;
wherein the tip comprises a cylindrical part and a tapered part extending from the cylindrical part to an outlet; the cylindrical part being attached to the moving part; the cylindrical part comprising a central channel surrounded by at least two peripheral channels; the central channel and the peripheral channels extending across the cylindrical part of the tip wherein the central channel comprises a pellet together with a foldable retainer in a folded position; the first peripheral channel comprising an opening at the tapered part, the first peripheral channel comprising a first dissection tool that is capable to be protruded from the tip; a blinded peripheral channel comprising a location sensor configured to be detected by an external localizer for locating continuously the tip.

The invention is advantageously completed by the following features, taken alone or in any technically possible combination thereof:
the cylindrical part comprises a second peripheral channel comprising a second dissection tool that is capable to be protruded from the tip, the second dissection tool being adapted to implement a dissection different to the one implemented by the first dissection tool;
the first dissection tool is an electrosurgery dissection tool and the second dissection tool is a hydro-dissection tool, the second peripheral channel are adapted to bring a hydro jet coming from a hydro jet generator connected to the laparoscopic tool;
the electrosurgery dissection tool comprises an electrode protruding from a centering piece that is folded in the first peripheral channel and is unfolded outside the first peripheral channel, the electrosurgery electrode being disposed on a top of the centering piece so that the electrosurgery electrode is always centered relative to the tip when unfolded, the electrosurgery dissection tool being adapted for extending the electrosurgery electrode outside the tip and for retracting the electrode surgery electrode inside the first peripheral channel;
it comprises, at the proximal end, a first handle adapted to operate the first surgical tool and a second handle adapted to control a release of the pellet;
it comprises, at the proximal end, a knob adapted to control the steering of the tip;
the knob is adapted to maintain the steering of the tip without any locking mechanism;
the shaft is rigid and comprises a cavity extending across the shaft from the proximal end toward the distal end, the cavity housing guiding mechanisms for controlling each of the surgical tool and for controlling a release of the pellet;
the foldable retainer is configured to be folded inside the central channel and to be deployed when moving outside the tool so that it prevents the pellet to return back to the tool after its release.

The tool is capable of achieving the following tasks without extraction and reinsertion of the tool in the patient:
Continuous localization of the tip of the tool preferably relative to external clues, irrespective of the bending of the tip of given tool;
Performing an opening of an area (for instance peritoneal fascia) using electrosurgery dissection while preserving the underneath structure;
Dissection of the connective tissue around the region to be treated (for instance around the portal vein structure) while preserving the nervous tissue or other delicate structure using hydro-dissection
Inserting the pellet containing the bioactive molecule in the lodge created by the previous step;
Unfolding of a restrainer fixed at the distal part of the pellet to prevent pellet extraction.

According to an embodiment, the tool is a single-use tool and made partially in surgical grade plastic and prior to the insertion in a cavity it needs to be connected to:
a known electrosurgical generator that can be activated by a foot pedal;
a hydro jet generator that can be manually or electrically powered since the dissection volume is limited to less than 20/30 cc (for the size of the lodge expected in this application) and because the dissection itself is done on weak connective tissue.
an internal electromagnetic localizer attached to steerable tip and working in conjunction with a reference sensor located on the patient, an electromagnetic field generator and software suitable for analyzing the signals of the mobile and reference sensor in respect of a computed tomographic image of a region to be treated.

Operating the surgical tool is done using hand-triggers handles allowing extraction and retraction of the various components of the tool together with hand-activated knob used for steer-ability of the tip of the tool. The tool is advantageously introduced into the cavity of the patient by means of a laparoscopy cannula and since the tool is capable, through the motion of the shaft of the laparoscopy cannula of significant movement relative to the region to be treated, the steer-ability of the tip is limited to two dimensions simplifying as such the mechanism supported by the knob as mentioned earlier.

In a preferred embodiment, the present disclosure relates to a laparoscopic tool designed to insert a pellet of a bioactive molecule in the vicinity of the portal vein wall, the bioactive molecule being developed to restore the potency of the portal glucose sensor, which is markedly impaired in individuals with insulin resistance. The tool facilitates the insertion of the pellet within a precise area, difficult to reach using laparoscopy and with potential adverse surgical outcomes.

### BRIEF DESCRIPTION OF THE DRAWINGS

Other features and advantages of the present disclosure will appear in the following detailed description. Embodiments of the invention will be described with reference to the drawings:

FIG. 1 illustrates a tool overall architecture according to an embodiment;

FIG. 2 illustrates a tip of the tool according to an embodiment;

FIG. 3 illustrates an electrosurgical electrode in position (top) and the release of the pellet (bottom) according to an embodiment;

FIG. 4 illustrates the insertion of the pellet and the associated method for refraining self-extraction of the pellet according to an embodiment;

FIG. 5 illustrates the steps of a method for releasing a pellet according to an embodiment.

### DESCRIPTION

### General Description of the Tool

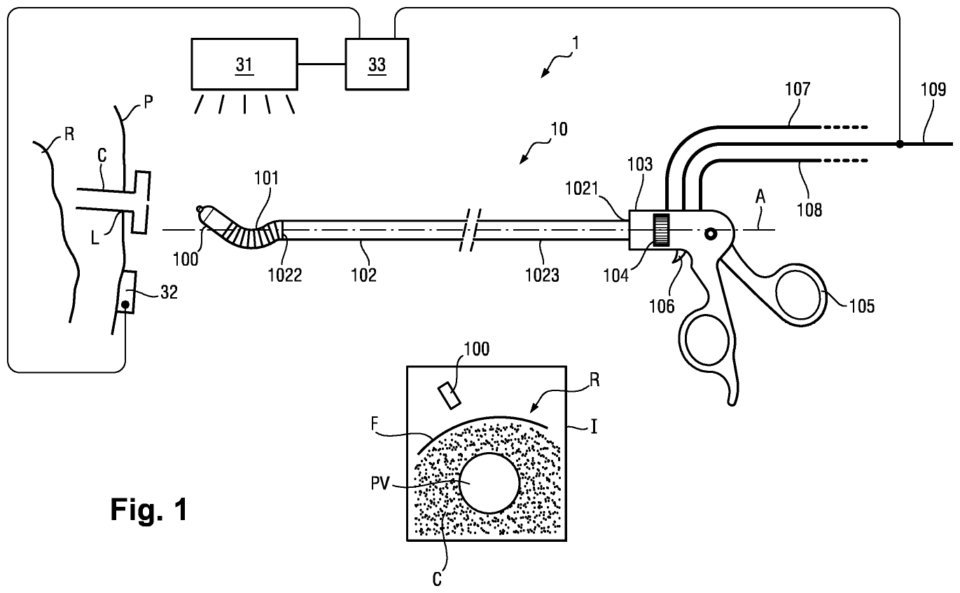

FIG. 1 illustrates schematically a surgical 1 scene wherein a pellet needs to be inserted in a region R to be treated of a patient P. For instance, as it will be explained in detail in the following, this region is at the vicinity of the portal vein for treating insulin resistance and/or restoring glucose homeostasis in the patient P. The region R to be treated is accessed by a laparoscopic cannula C introduced in the patient P. This cannula C defines a lumen L for the introduction of a laparoscopic tool 10 integrating several functionalities. In order to control the displacement of the laparoscopic tool 10, an image I of the region to be treated is displayed and the laparoscopic tool 10 is located in real time in relation to this image I. Thus, the surgery visualized the tool along the image of the region to be treated for accurate access to this region.

This figure depicts the laparoscopic tool 10 overall architecture comprising at the left of the tool 10 a tip 100 attached to a steerable part 101 (or a moving part) which is attached to a shaft 102 and, in the right of the tool 10, a control set 103 from which extend several connections 107, 108, 109 for connecting the tool 10 for interaction with the surgeon (not shown) and including several actuators 104, 105, 106 to be actuating by the handle of the surgeon.

The shaft 102 comprises a proximal end 1021 close to the surgeon and a distal end 1022 intended to be introduced in the lumen L formed in the patient P by means of the laparoscopy cannula C. The tip 100 comprises several functionalities that can be controlled by the surgeon. These functionalities replace several tools for each function.

In particular, the tool 10 comprises at least one dissection tool, preferably two dissection tools for accessing the region to be treated and a pellet to be released in the region. When the tool 10 comprises two dissection tools, each dissection tool is configured to implement a particular type of dissection.

The laparoscopic tool 10 is preferably a single-use tool made partially in surgical grade plastic as it also comprises also metallic parts for instance for the connection of the tool. Also, the tool 10 is advantageously stored in a sterile envelope and needs to be connected prior the insertion in the patient.

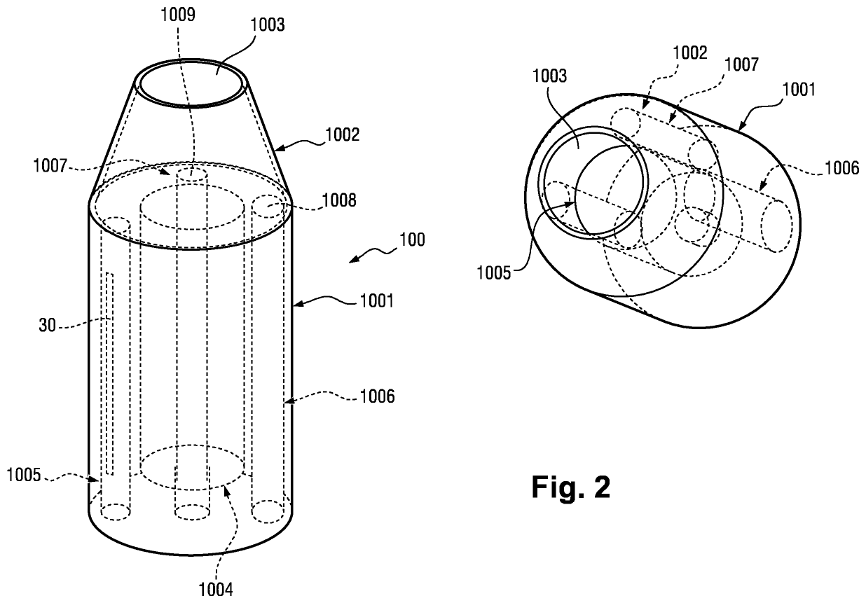

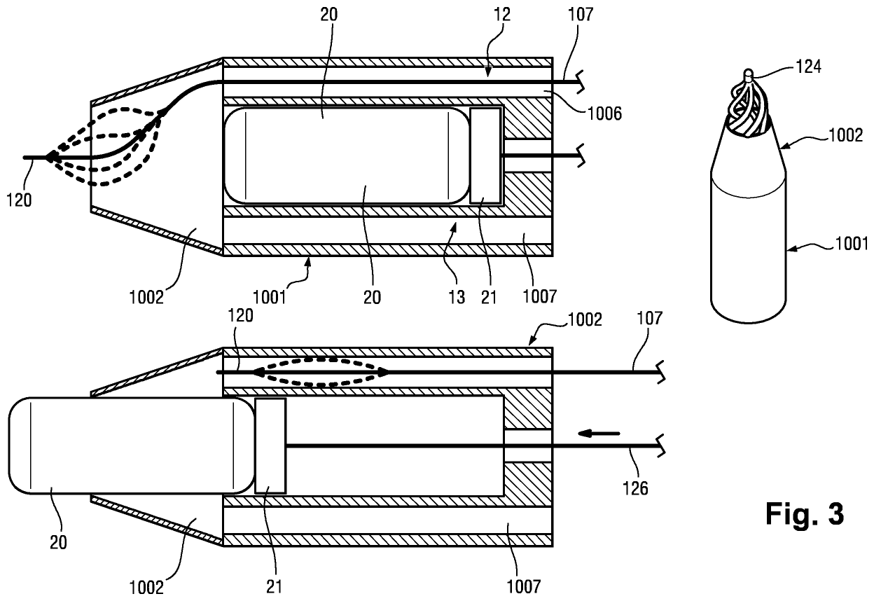

As illustrated on FIG. 2 and FIG. 3, the tip 100 comprises a cylindrical part 1001 and a tapered part 1002 extending from the cylindrical part 1001 to an outlet 1003. The cylindrical part 1001 is attached to the moving part 101 and comprises a central channel 1004 surrounded by at least two peripheral channels, preferably three peripheral channels 1005, 1006, 1007. The central channel 1004 and the peripheral channels 1005, 1006, 1007 extend across the cylindrical part 1001 of the tip 100. The channels of the tip 100 are in communication with corresponding channels in the shaft 102.

One peripheral channel is a blind peripheral channel 1005 and comprises a location sensor 30 configured to be detected by an electromagnetic field generator 31 acting as an external localizer for locating continuously the tip 100 relative to a reference sensor 32 located on the patient P. The location is therefore made relative to this reference sensor 32 even if the patient is moving. In particular, the location sensor 30 is an electromagnetic localizer. This location sensor 30 is connected to a processing unit 33 (connected to a computer, not shown) by a connection 109 extending through the shaft 102. This processing unit 33 comprises a software suitable for analyzing the signals of the tool tip location sensor 30 and to detect the reference sensor 32. The processing unit is also connected to the external electromagnetic field generator 31. The location is made in relation with a computed tomography image of the region to be treated of the patient so that, during the intervention, the tip 10 is displayed in real time. The use of an electromagnetic sensor allows an accurate location of the tip which is steerable.

The central channel 1004 contains a pellet 20 together with a folded retainer 21 attached to a release wire 126 permitting to control the release of the pellet into the patient. The pellet 20 has for instance a length of 10 mm and a diameter of 4.6 mm. The release of the pellet 20 is achievable by a release wire 126 located in the central channel 1004 that can be activated by a first handle 106 at the proximal end 1021 of the tool 10. The diameter of the central channel 1004 is adapted to the size of the pellet 20. The length of the release wire 126 permits to release the pellet 20 and the retainer 21. The retainer 21 prevents the pellet 20 to return back to the tool 10 after its extraction from the tool 10 and its release into the patient. In particular, the retainer 21 is designed so that it remains folded until it gains its resting size preventing extraction of the pellet 20 from the region to be treated. The retainer 21 has a foldable shape consisting in a memory capable wire and can be stored in a small volume.

A first peripheral channel 1006 comprises an opening 1008 at the tapered part 1002 and comprises a first dissection tool 12 that is capable to be protruded from the tip 100. In a preferred embodiment, a second peripheral channel 1007 comprises an opening 1009 and houses a second dissection tool 13 that is capable to be protruded from the tip 100.

The first dissection tool 12 is an electrosurgery dissection tool and the second dissection tool 13 is a hydro-dissection tool. These dissection tools permit to implement different types of dissection depending on the area to be dissected. In particular, the type of dissection depends on the robustness of the area to be dissected, the anatomical structures surrounding the area to be dissected, etc. For instance, the fascia F (consisting in the mesenteric part of the peritoneum) is robust and can be dissected with the electrosurgery dissection tool while the connective tissue can be dissected with hydro-dissection tool. Furthermore, the hydro-dissection tool is used when vital structures are present around the area to be treated. This is in particular the case for the dissection of the connective tissue around the portal vein that must not be damaged.

According to an embodiment, the electrosurgical dissection tool 12 is capable to be protruded from the first peripheral channel 1006. According to an embodiment, the electrosurgical dissection tool is initially outside the peripheral channel 1006 and can only be pulled inside the peripheral channel 1006 after its use. The electrosurgical dissection tool 12 comprises an electrosurgery electrode 120 with its insulation protruding from a centering piece 121 that is folded when located into the first peripheral channel 1006 and unfolded outside the first peripheral channel 1006. The electrosurgery electrode 120 is disposed on the top 124 of the centering piece 121 so that the electrosurgery electrode 120 is always centered relative to the tip 101 when unfolded. The electrosurgery electrode 102 is connected by means of a connection 107 to a classical electrosurgical generator activated by a foot pedal (not shown). Furthermore, a second handle 105 is connected to the electrosurgery electrode 120 and permits to expend and retract it from and into the tool.

The first peripheral channel 1006 is larger than the second peripheral channel 1007. The second peripheral channel 1007 is thus smaller than the first peripheral channel 1006 and is dedicated to the hydro-dissection tool and in particular to the hydro jet flow coming from a hydro jet generator connected to the tool 10 by means of a flexible tube 108.

The tapered part 1002 of the tip 100 serves for centering the various extractable elements containing in the central and peripheral channels. The internal side of the tapered part 1002 is hollow to facilitate the expansion or the retraction of the centering piece 121 of the electrosurgical electrode and the partial extension of the retainer 21. The opening 1003 of the tip 10 is of the size of the pellet preferably with a 100 μm difference to facilitate insertion in the region to be treated.

The tool 10 comprises at the proximal end 1021 a knob 104 for controlling the steering of the tip 100 since the moving part 101 is controlled by the knob 104. For instance, the tip 100 is attached to the moving part 101 by virtue of two wires winding and unwinding on the same reel after motion of the knob 104 which is directly connected to the reel or through a 90° mechanism so that the knob 104 is designed at 90° of a longitudinal axis of the shaft 102, the shaft 102 extending along this longitudinal axis. The friction of the knob 104 is designed so that after the motion of the knob 104, it maintains by himself the angle imposed on the moving part 101 without any locking mechanism. In one embodiment, the moving part 101 consists in a classical intermingled half cured tube made of flexible plastic suitable for a one-dimension motion that is preferably in direction perpendicular to the longitudinal axis AA.

The shaft 102 is preferably rigid and comprises a cavity with six sub-channels-two for the wires used for steer-ability of the tip, one for the movable electrosurgery electrode, one for the hydrojet dissection channel, one for the wire used to get data of the position of the location sensor and one for the wire used for extraction of the pellet and its retainer such as a umbrella-like retainer.

At the surgeon side the tool 10 comprises a casing 103 preferably made of plastic and connected to the shaft 102 and holds all the connection and the mechanism for the steerable tip, and the handles 105, 106.

On FIG. 3 the extension and retraction capabilities of the electrosurgical electrode (top part) and the extension procedure for the pellet and retainer in a form of a restraining umbrella (bottom part) are illustrated. The electrosurgical electrode 120 is capable to be protruded after activation of the second handle 105 of the tool 10. Since the electrode 120 is significantly of smaller diameter than the pellet 20, it needs to be centered using the centering piece 121 in a form of flexible cage that expands due to the elasticity of the plastic wires (memory capable) once it is not anymore stored in the body of the tool (see bottom part of FIG. 3). The size of the centering piece 121 is preferably a compromise between that needed for adequate centering and that necessary for adequate surgeon vision of the electrode tip 120.

Once the tip 100 of the tool 10 is inserted in region to be treated and after the hydro careful dissection of the connective tissue, the pellet 20 is extracted from its storage container together with its folded inverted umbrella 21.

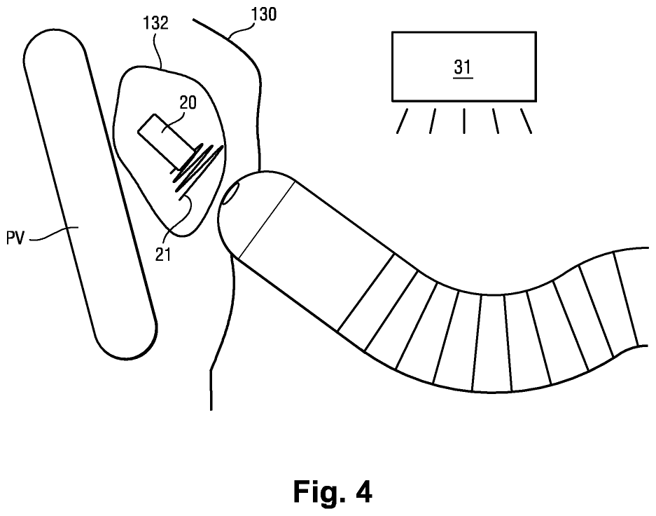

FIG. 4 illustrates the step of pellet insertion prior removal of the instrument. The pellet 20 is inserted in a region 130 to be treated (for instance at the vicinity of the portal vein PV) and preferably in the connective tissue C around this region within a volume 132 created by hydro jet dissection. This is achieved by the partial insertion of the tip of the tool in the region to be treated after the application of electrosurgical current while the electrode tip is protruding. The extraction of the pellet is associated with the unfolding of the retainer 21 which is preferably an inverted umbrella anchor that preferably consists in a memory capable wire. The spiral shape of the anchoring wire explains that it expands both perpendicular to the pellet and within the great axis of the pellet. This achieves an adequate anchoring while allowing a limited storage volume suitable for a laparoscopy instrument.

### Method for Releasing a Pellet With the Laparoscopic Tool

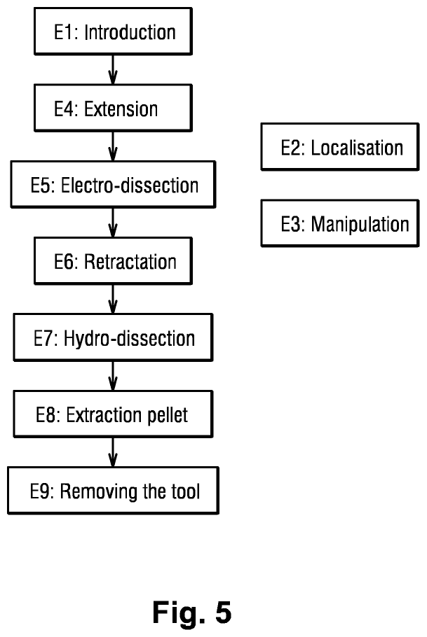

In operation and in relation with FIG. 5, the surgeon introduces in a laparoscopic cannula the tool 10 as described above (step E1). During the intervention the tip 100 of the tool 10 is continuously located (step E2) and the tool 10 is manipulated by means of the handle 105 and the knob 104 for positioning the tip 10 (step E3) accurately.

Once the tool 10 is introduced at the vicinity of the region to be treated, the surgeon controls the extension of the electrosurgical electrode 120 (see FIG. 3, top part) (step E4) and triggers a dissection of the tissues surrounding the region to be treated while handling the tool 10 by means of the handle 105 (step E5). Alternatively, the electrosurgical electrode 120 is already outside the tip 100 and step E4 is therefore not implemented. For instance, the fascia F (see FIG. 1) is dissected by means of the electrosurgical electrode 120.

Once the tissues surrounding the region are dissected, the surgeon retracts the electrosurgical electrode 120 (step E6, see FIG. 3, bottom part).

Then, the surgeon triggers the hydro dissection of the connective tissue C (see FIG. 1) for defining a region in which the pellet needs to be inserted (step E7). As can be seen on FIG. 1, the connective tissue C surrounds the portal vein PV.

After the hydro dissection of the connective tissue, the region to be treated can be accessed, and the surgeon triggers the extraction of the pellet 20 by means of the handle 105 (step E8 and see FIG. 4).

The tool 10 is then removed (step E8).

### Use of the Tool for Inserting Pellet-Releasing Bioactive Compounds at the Vicinity of the Portal Vein for Treating Insulin Resistance and/or Restoring Glucose Homeostasis in a Subject in Need

The glucose homeostasis after a meal depends on the integration of glucose-sensing mechanisms in the portal vein (Soty et al., 2017). This sensor is markedly impaired in a preclinical model of insulin resistance due to reduced expression of GLP-1r receptor, which is essential for glucose sensing (Malbert et al, 2021). Restauration of GLP-1r density is feasible using locally administered dihydrotestosterone at a concentration of 1/10 or less of the parenterally administered hormone. More importantly, the restauration of the GLP-1r density at the periportal level is associated with the recovery of insulin sensitivity in the same preclinical model of insulin resistance.

Several surgical steps are mandatory to achieve the positive outcome of the treatment obtained using locally administered bioactive molecule, all of which require sequential insertion of dedicated laparoscopic tools. Furthermore, since the portal structure in need of GLP-1r restoration is narrow, the entire procedure involves image guidance of the tip of the instruments, a solution that prevents using flexible laparoscopy instruments since the uncertainty of the tip location is far too considerable. This renders the insertion of the pellet containing the bioactive molecule somewhat technically demanding since the portal structure and the instrument shaft is almost parallel (or with a limited angle relative to each other), precluding an easy insertion of the tip of the forceps that hold the pellet. An additional pitfall in periportal pellet insertion relates to creating a surgical lodge suitable for inserting the pellet. This procedure needs the use of a dissector, the tip of which is not easy under direct vision, with potentially harmful consequences, especially with a fragile structure such as the portal vein. The somewhat blind dissection is also probably damageable for the intricate nervous network lying alongside the connective tissue surrounding the portal structure. Finally, the movements of the portal system associated with changes in stomach and intestine position after the meal are strong enough to extract the pellet from its surgically prepared lodge. The closure of the internal peritoneal layer supporting the periportal connective tissue is, therefore, mandatory. This is tricky using surgical stitches that cannot hold adequately onto the otherwise minute peritoneal layer. The alternative option used so far was to stick, using surgical glue, a piece of dacron mesh blocking the peritoneal hole hence preventing the unwanted extraction of the pellet.

The use of a laparoscopy cannula that allowed large bore (up to 30 mm) shaft without significant gas leakage together make possible the use of the single-use laparoscopic tool as described above.

The present disclosure as explained aims at providing a single surgical tool suitable for laparoscopy and capable of achieving the following tasks without extraction and reinsertion in the abdominal cavity:
Continuous localization of the tip of the relative to external clues, irrespective of the bending of the tip of the given instrument;
Opening of the peritoneal fascia using electrosurgery desiccation while preserving the underneath structure;
Dissection of the connective tissue around the portal structure while preserving the nervous tissue using hydro-dissection. This is made possible by inserting the tip of the instrument (after electrosurgery tip retraction) into the breach in the peritoneal fascia created during the former step;
Inserting the pellet containing the bioactive molecule in the lodge created by the previous step;
Opening of a restrainer fixed at the distal part of the pellet to prevent pellet extraction.

### REFERENCES

Soty et al., 2017: Soty, M., Gautier-Stein, A., Rajas, F., & Mithieux, G. (2017). Gut-Brain Glucose Signaling in Energy Homeostasis. Cell Metab, 25(6), 1231-1242.
Malbert et al, 2021: Malbert, C.-H., Chauvin, A., Horowitz, M., & Jones, K. L. (2020). Glucose-Sensing Mediated by Portal GLP-1 Receptor is Markedly Impaired in Insulin-Resistant Obese Animals. Diabetes.

1. A laparoscopic tool (10) comprising:
a shaft (102) comprising a proximal end (1021) and a distal end (1022);
a tip (100);
a moving part (101) disposed between the distal end (1022) of the shaft (102) and the tip (100), the moving part (101) being attached to both the distal end (1022) and the tip (100), the moving part (101) being adapted to allow movement of the tip (100) relative to the shaft (102);
wherein the tip (100) comprises a cylindrical part (1001) and a tapered part (1002) extending from the cylindrical part (1001) to an outlet (1003); the cylindrical part (1001) being attached to the moving part (101); the cylindrical part (1001) comprising a central channel (1004) surrounded by at least two peripheral channels (1005, 1006, 1007); the central channel (1004) and the peripheral channels (1005, 1006, 1007) extending across the cylindrical part (1001) of the tip (100) wherein the central channel (1004) comprises a pellet (20) together with a foldable retainer (21) in a folded position; the first peripheral channel (1006) comprising an opening (1008) at the tapered part (1002), the first peripheral channel (1006) comprising a first dissection tool (12, 13) that is capable to be protruded from the tip (100); a blinded peripheral channel (1005) comprising a location sensor (30) configured to be detected by an external localizer (31) for locating continuously the tip (100).

2. The laparoscopic tool of claim 1, wherein the cylindrical part comprising a second peripheral channel (1007) comprising a second dissection tool (12, 13) that is capable to be protruded from the tip (100), the second dissection tool (13) being adapted to implement a dissection different to the one implemented by the first dissection tool.

3. The laparoscopic tool of claim 2, wherein the first dissection tool is an electrosurgery dissection tool (12) and the second dissection tool is a hydro-dissection tool (13), the second peripheral (1007) channel being adapted to bring a hydro jet coming from a hydro jet generator connected to the laparoscopic tool.

4. The laparoscopic tool of claim 3, wherein the electrosurgery dissection tool (12) comprises an electrode (120) protruding from a centering piece (121) that is folded in the first peripheral channel (1005) and is unfolded outside the first peripheral channel (1005), the electrosurgery electrode being disposed on a top (124) of the centering piece (121) so that the electrosurgery electrode (120) is always centered relative to the tip (100) when unfolded, the electrosurgery dissection tool being adapted for extending the electrosurgery electrode (120) outside the tip (100) and for retracting the electrode surgery electrode (120) inside the first peripheral channel.

5. The laparoscopic tool of claim 1, comprising at the proximal end (1021) a first handle (105) adapted to operate the first surgical tool and a second handle (106) adapted to control a release of the pellet (20).

6. The laparoscopic tool of claim 1, comprising at the proximal end (1021) a knob (104) adapted to control the steering of the tip (100).

7. The laparoscopic tool of claim 6, wherein the knob (104) is adapted to maintain the steering of the tip without any locking mechanism.

8. The laparoscopic tool of claim 1, wherein the shaft (102) is rigid and comprises a cavity (1023) extending across the shaft (102) from the proximal end (1021) toward the distal end (1022), the cavity (1023) housing guiding mechanisms for controlling each of the surgical tool and for controlling a release of the pellet.

9. The laparoscopic tool of claim 1, wherein the foldable retainer (21) is configured to be folded inside the central channel and to be deployed when moving outside the tool so that it prevents the pellet to return back to the tool after its release.
